(19) United States
(12) Patent Application Publication
(10) Pub. No.: US 2020/0078206 A1
(43) Pub. Date: Mar. 12, 2020

(54) Protection Device for Ostomy Pouch

(71) Applicant: ZONI LLC, San Francisco, CA (US)

(72) Inventor: John Chiladakis, Davis, CA (US)

(21) Appl. No.: 16/669,377

(22) PCT Filed: May 5, 2017
(86) PCT No.: PCT/US17/31173

§ 371 Date: Oct. 30, 2019

(51) Int. Cl. A61F 5/449

(52) U.S. Cl. CPC A61F 5/449

(57) ABSTRACT

The disclosed device is designed to be worn by ostomy patients. It is designed to be worn between the body of the patient and a garment of the patient. The device comprises a base plate, a bracket and a belt clip, with a stoma pouch passing between the base plate and the bracket. One side of the bracket is attached to the base plate, and the second side of the bracket attached reversibly to the base plate by a snap latch. The advantages of the use of the device include an improvement of the patient's quality of life, specifically the ability of the patient to wear his or her own clothing in a manner that is similar to those without a stoma.

### FIELD OF THE INVENTION

The present invention is directed to medical devices. More particularly, the invention relates to the field of wearing ostomy pouches.

### DESCRIPTION OF RELATED TECHNOLOGY

One of the many problems faced by a patient with a stoma is wearing everyday clothing in a manner to which the patient is accustomed. A stoma is most frequently located above the waistband. In order not to put pressure on the stoma pouches, or not to pinch the stoma pouches by the belts or beltlines of pants, many ostomates wear the top of the pants above their stoma. This mitigates the problem of the pressure on the pouch and the problem with pinching the pouch, but it pushes the top of the pants significantly above the waistline. This is a problem because wearing pants in manner for which the pants were not designed may be uncomfortable, embarrassing and even unsafe.

A stoma protective cap is disclosed in U.S. Patent Application Publication No. 2009/0182191. The stoma protective cap for protection of the stoma, encompassing a plate which has a curvature, which curvature is formed such that it spans a cavity across the stoma and the plate encompasses at least one retaining region which enables a fastening on clothing so that the plate can be securely held between the clothing and the body.

A colostomy protector is disclosed in U.S. Pat. No. 2,314,724. The patent teaches a surgical appliance embodying a non-yielding cup-shaped body having a rear opening and adapted to surround and shield a colostomy; a hollow stub integral with said body and communicating with the lower portion thereof; a groove in the edge of the body wall surrounding said openings, said groove having less width tan the thickness of the wall and adapted to form a vacuum seal between said body and the skin tissue around the colostomy; and a flexible sack detachable mounted on said hollow stub.

### SUMMARY OF THE INVENTION

The present invention relates to a device designed to be worn by ostomy patients. This device is designed to be worn between the body of the patient and a garment of the patient. The device comprises a base plate, a bracket and a belt clip. A stoma pouch passes between the base plate and the bracket.

The advantages of the present invention is the improvement of the patient's quality of life. Specifically, advantages include the ability of the patient to wear his or her own clothing in a manner that is similar to those without a stoma; the ability of the patient to insure that the stoma pouch or a catheter is not pinched during the wearing of clothing; and the ability of the wearer to pass a tubing that may be attached to the stoma pouch through the typical zippered opening located in the front of many pants.

The device of the present invention is designed to be worn by an ostomy patients. The device allows a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising a bracket outer surface, a bracket inner surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket. The device may comprise other parts in addition to ones recited in subparagraphs (a) through (c) above.

The base plate faces are substantially rectangular, having either sharp corners, rounded, or a combination thereof. The top plate edge may be straight, or it may comprise a concavely curved portion. The concavely curved portion may be more comfortable to wear.

The base plate may also have a plurality of holes connecting the front plate surface to the back plate surface. Such holes may be any size and shape, as long as their size and shape is sufficiently consistent with the function of the base plate. One advantage of holes being round, oval or any other shape wherein the hole wall does not have any sharp concave corners, is that it is easier for the back plate to be exposed to stresses without tearing the back plate.

The size of the base plate is selected so that it matches the size of the bracket, which in turn is selected to match the size of the ostomy pouch. Depending on the material, according to some embodiments the base plate is flexible, so that when the bracket is unlatched from the base plate the base plate, the base plate folds away from the bracket.

The bracket is a C-shaped piece that is permanently attached to the base plate on one side, and attaches on the other side by a latch. The latch is designed to be opened and closed with the latch.

The latch is comprised of two arm portions and a linking portion. The inside corners of the latch as well as corners formed by the latch and the base plate may be either sharp, or they may be filleted, or some of them may be sharp and other filleted. The outside corners of the bracket may be sharp or they may be rounded. It is generally advantageous for the outer corners to be rounded and the inner corners to be filleted, due to stresses distribution, not tear as easily, not tearing garments or patients.

The second arm portion comprises a first snap latch portion which is designed to mate with the second snap latch portion that is affixed on the base plate. The first snap latch portion and the second snap latch portion together form a snap latch. The snap generally occurs when the device is placed in the wearing position, and the patient then snaps the latch in place.

The two snap latch portions may be manufactured from the same material or from a different material, as long as at least one of them is flexible enough so that the male snap latch portion snaps reversibly into the female snap latch portion.

The device may have the latch on either side of the base plate. The device may be a left swinging device or a right swinging device.

Under one embodiment the ridge of the male snap latch portion along the vertical direction is uniform. Under another embodiment the head diameter varies along the vertical direction. Under one embodiment the male snap latch portion comprises a fin. The fin is a projection from the male snap latch portion that when the snap latch is closed, the fin fits into slot in the female snap latch portion. There may be more than one fin on the ridge and a complementary number of slots in the female snap latch portion. The presence of the fin or fins helps to stabilize the closure of the snap latch.

The device further comprises one or more belt clips. The sides, top and bottom of the belt clip may be straight or curved, they may be straight, leaning in or leaning out. The corners of the belt clip may be sharp of rounded.

The structure of the device may be modular or unibody. For a modular construction, the device may be formed from plastic, metal, wood, or other rigid material or semi-rigid material, and from a flexible material such as rubber, leather, or plastic.

Under one embodiment the device has a unibody structure, wherein the device composed of a single material which has uniform chemical and physical properties. The material used is rigid enough that the device holds its shape during patients everyday activities to allow for the ostomy pouch to be passed therethrough. However, the material used is also flexible enough that the latch can be opened and closed.

Under one embodiment the material from which the unibody device of the present invention is formed and that exhibits such properties is a polymer. The device may be formed by injection molding, or by other means. Suitable polymers also include ethylene vinyl acetate, polyethylene-octene and polyethylene-butene.

Under one embodiment the polymer, co-polymer, or mixtures thereof further comprises other ingredients that improve the physical properties of the material, chemical properties of the material, and/or aesthetic properties of the material. Possible ingredients include a dye, a lubricating agent, a nucleating agent, an antioxidant, an antistatic agent, and any combinations thereof.

At least a part of the surface of the device of the present invention may be coated. The device may comprise several coats or coatings of portion of the device.

The device of the present invention may further comprise a label, such as an adhesive label. The device may have more than one label. A label may be affixed permanently or temporarily. Such a label may be used to communicate important information to the patient such as instructions on wearing the device, reminders to the patient, or warnings related to the use of the device.

The device of the present invention has been specifically designed to be worn by an ostomate patient between the patient's skin and clothing to allow for a free passage of the pouch. With the device of the present invention, the patient is able to wear the patient's pants, skirt or other lower body garment at the customary height. The device is designed to be attached to a portion of the garment by several means, such as by the belt clip. The device may be attached by the belt clip to an edge the garment or a fold in the garment, such as the top of the pants.

The device of the present invention is designed so that the ostomy pouch when worn is not pinched or restricted by the clothing. When the pouch and the device are worn, the pouch is located between the bracket and the base plate. The pouch is worn loosely, so that the pouch moves freely along the longitudinal direction, as the patient moves. The device of the present invention is also designed to work with a catheter.

The invention is defined by at least fifteen aspects.

In the first aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket.

In the second aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the first snap latch portion is a female snap latch portion, and the second snap latch portion is a male snap latch portion.

In the third aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the first snap latch portion is a male snap latch portion, and the second snap latch portion is a female snap latch portion.

In the fourth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge, wherein the top plate edge comprises a concavely curved portion; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket.

In the fifth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device, further comprising linking portion outer surface, a first arm portion outer surface, a second arm portion outer surface, a linking portion inner surface, a first arm portion inner surface, and a second arm portion inner surface; further comprising: (d)(i) a fillet formed by the linking portion inner surface and the first arm portion inner surface, (ii) a round formed by the linking portion outer surface and the first arm portion outer surface, (iii) a fillet formed by the linking portion inner surface and the second arm portion inner surface, (iv) a round formed by the linking portion outer surface and the second arm portion outer surface, (v) a fillet formed by the front plate face and the first arm portion outer surface, (vi) a fillet formed by the front plate face and the first arm portion inner surface, (vii) a fillet formed by the front plate face and the second snap latch portion inner surface, (viii) a fillet formed by the front plate face and the second snap latch portion outer surface, or (ix) any combination thereof.

In the sixth aspect, the present invention relates to a device for allowing a passage of an ostomy pouch between a garment and a patient's body comprising (a) a base plate comprising a front plate face, a back plate face, a top plate edge, a bottom plate edge, a first plate side edge, a second plate side edge, and a plurality of holes; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket.

In the seventh aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the first snap latch portion is a female snap latch portion, and the second snap latch portion is a male snap latch portion; or wherein the first snap latch portion is a male snap latch portion, and the second snap latch portion is a female snap latch portion; and wherein the male snap latch portion comprises a fin and the female snap latch comprises a slot that engages the fin.

In the eighth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein at least a portion of a surface of the device is covered by a coating.

In the ninth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device further comprises a label.

In the tenth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure.

In the eleventh aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure, and wherein the device is formed by injection molding.

In the twelfth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure, and wherein the device is comprised of material selected from the group consisting of ethylene vinyl acetate, ethylene-vinyl rubber, polyethylene, polyvinyl chloride, polyurethane, rubber, polypropylene, polyethylene terephthalate, high density polyethylene, low density polyethylene, polyethylene acrylonitrile butadiene styrene, high impact polystyrene, polypropylene, polycarbonate, acrylonitrile butadiene styrene, polyamide, polyethersulfone, a co-polymer of any one or more of the preceding, and a mixture thereof.

In the thirteenth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure, and wherein the device is comprised of material selected from the group consisting of ethylene vinyl acetate, ethylene-vinyl rubber, polyethylene, polyvinyl chloride, polyurethane, rubber, polypropylene, polyethylene terephthalate, high density polyethylene, low density polyethylene, polyethylene acrylonitrile butadiene styrene, high impact polystyrene, polypropylene, polycarbonate, acrylonitrile butadiene styrene, polyamide, polyethersulfone, a co-polymer of any one or more of the preceding, and a mixture thereof, wherein the material further comprises an ingredient selected from the group consisting of a pigment, an antimicrobial agent, a lubricating agent, a nucleating agent, an antioxidant, an antistatic agent, and any mixture thereof.

In the fourteenth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure, and wherein the hardness of the material from which the device is formed is between about 30 units and about 90 units as measured by ISO 868 under Shore A scale.

In the fifteenth aspect, the present invention relates to a device for allowing a passage of an ostomy pouch between a garment and a patient's body comprising (a) a base plate comprising a front plate face, a back plate face, a top plate edge, a bottom plate edge, a first plate side edge, and a second plate side edge; (b) a bracket comprising a linking portion, a first arm portion, and a second arm portion, further comprising an outer bracket surface, an inner bracket surface, a top bracket edge, a bottom bracket edge, wherein the first arm portion is attached to the front plate face, and wherein the second arm portion comprises a first snap latch portion that engages a second snap latch portion attached to the front plate face; and (c) at least one belt clip attached to the top bracket edge or the outer surface of the bracket; wherein the device has a unibody structure, and wherein the elastic modulus of the material from which the device is formed is between about 20 MPa and about 200 MPa as measured by ISO 527.

### BRIEF DESCRIPTION OF THE INVENTION

An exemplary embodiment of the invention is illustrated in the drawings and further explained hereinunder.

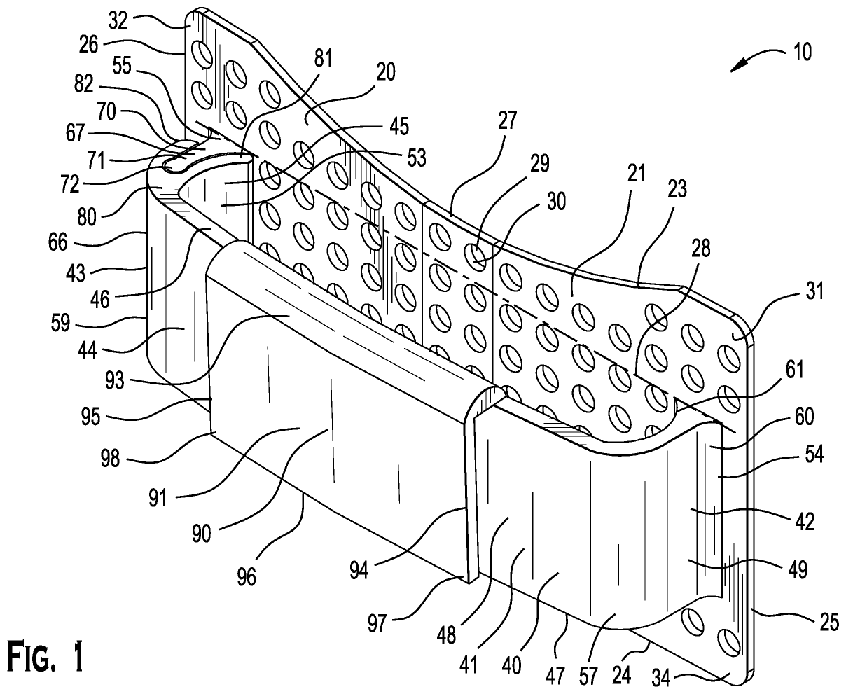

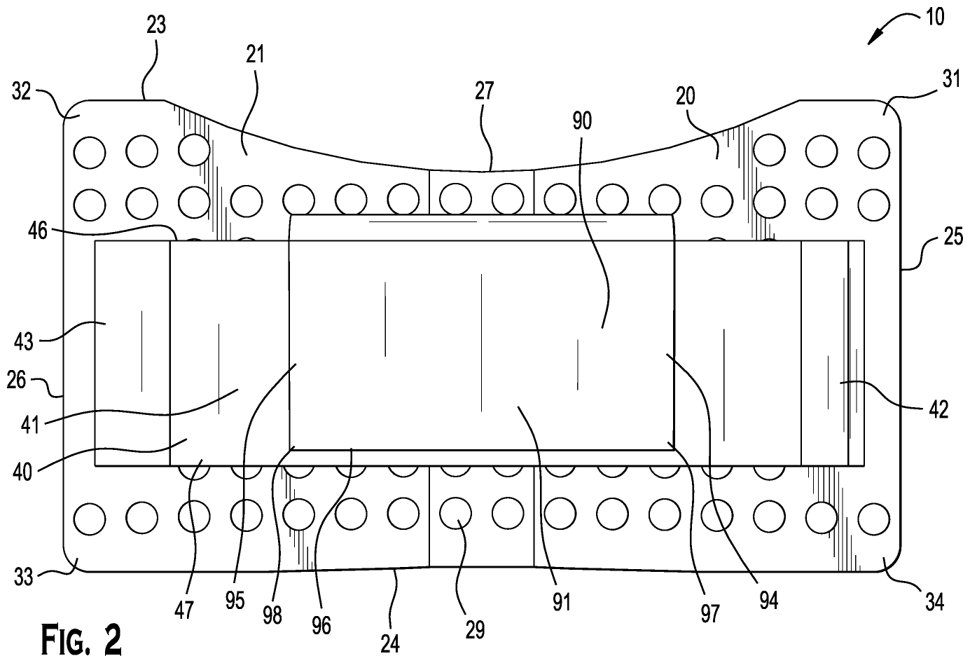

FIG. 1 is a perspective view of the device for allowing of a passage of an ostomy pouch.

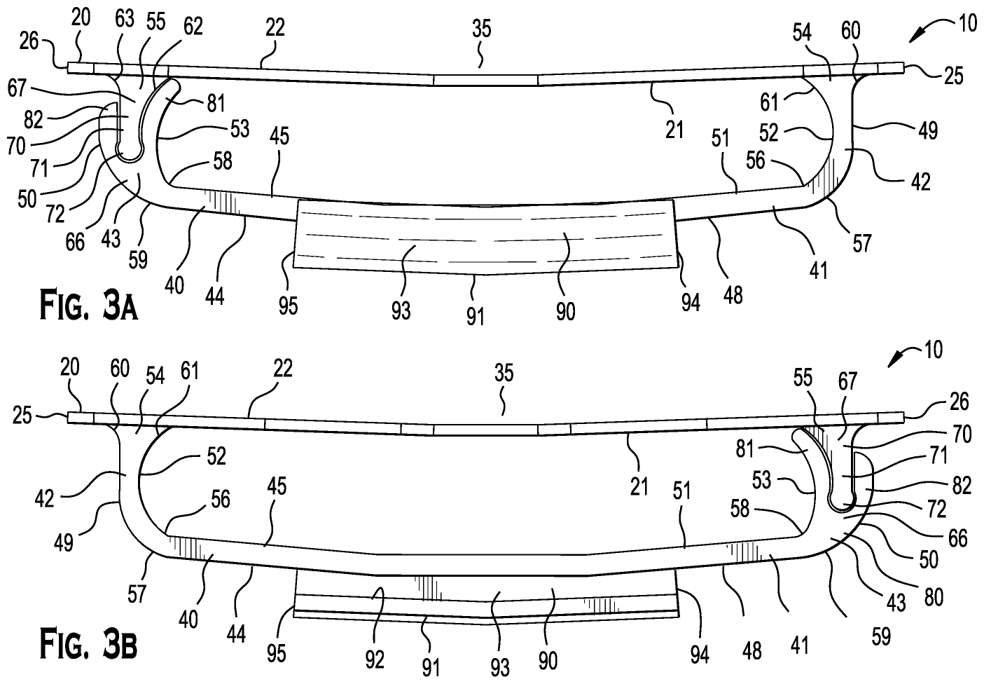

FIG. 2 is a front view of the device.

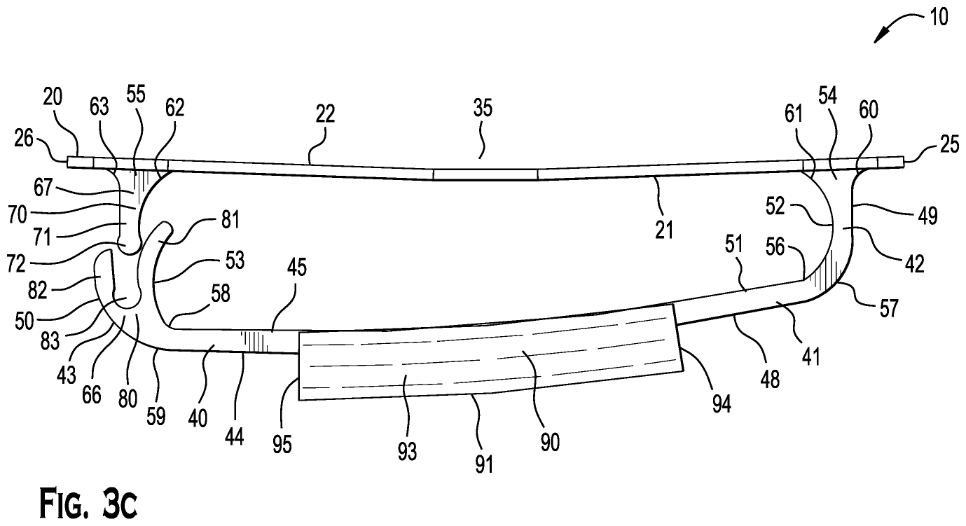

FIG. 3a is a top view of the device.

FIG. 3b is a bottom view of the device.

FIG. 3c is a top view of the device when the snap latch is open.

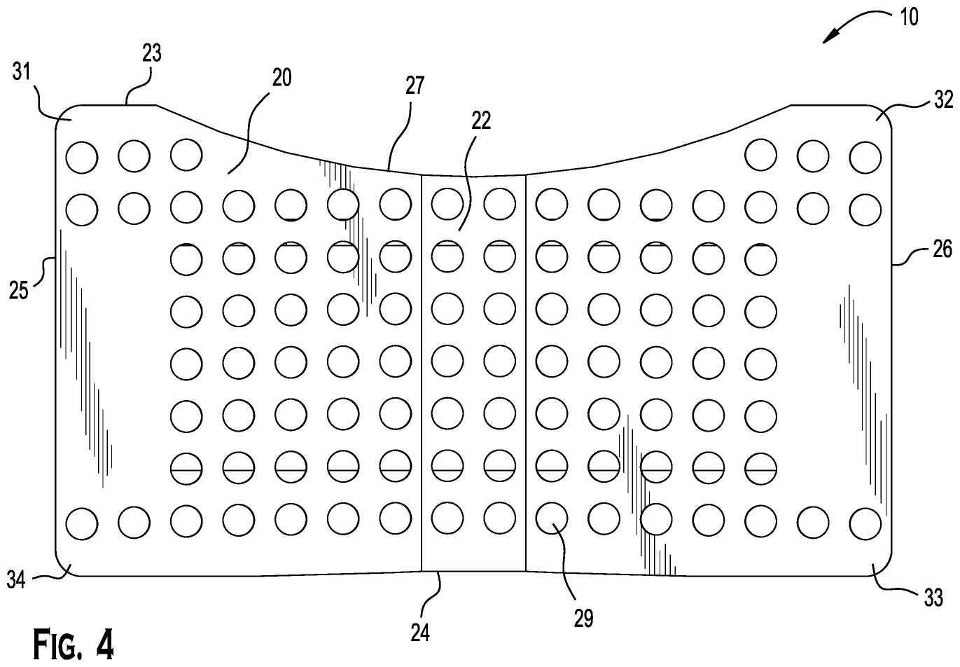

FIG. 4 is a back view of the device.

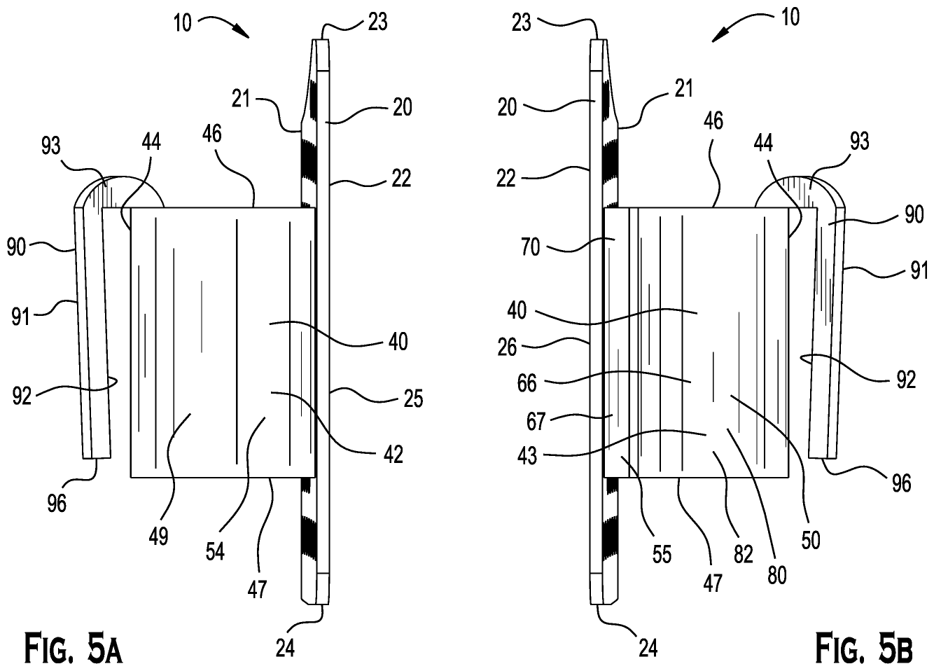

FIG. 5a is a side view of the device from the right.

FIG. 5b is a side view of the device from the left.

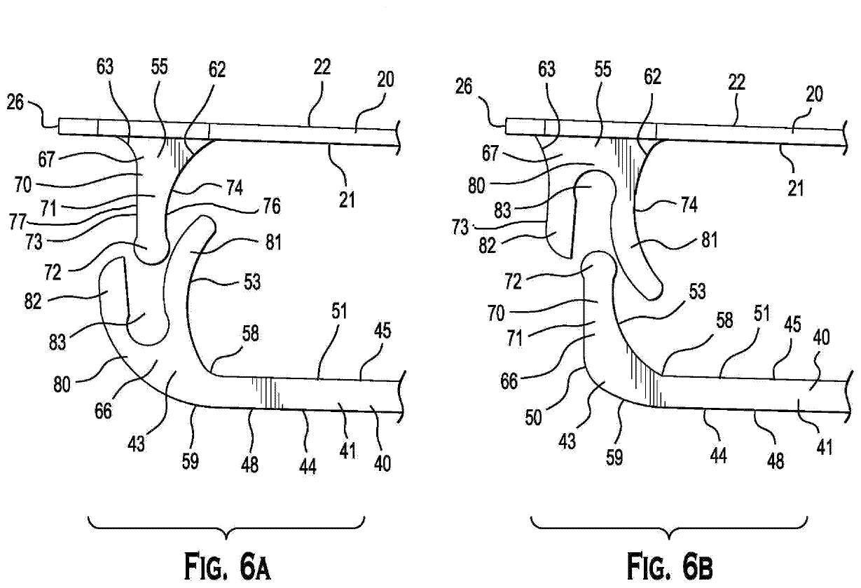

FIG. 6a is a top view of the snap latch wherein the second arm comprises a female snap latch portion.

FIG. 6b is a top view of the snap latch wherein the second arm comprises a male snap latch portion.

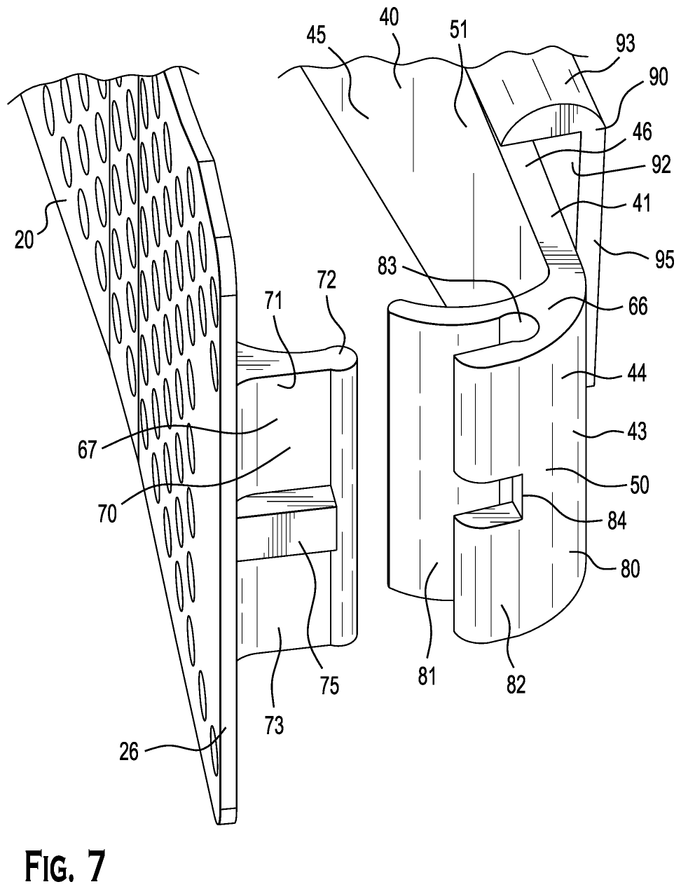

FIG. 7 is an axonometric view of the latch with a fin.

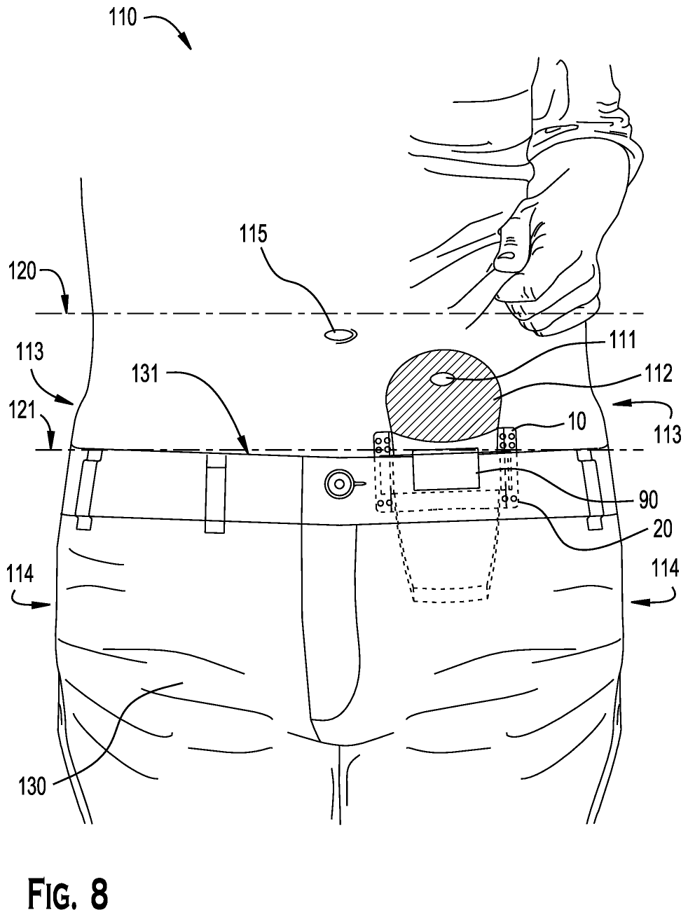

FIG. 8 is a view of an ostomy pouch and the device as worn by a patient.

### DETAILED DESCRIPTION OF THE INVENTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. It is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The singular form of any class of the ingredients refer not only to one chemical species within that class, but also to a mixture of those chemical species; for example, the term “polymer” in the singular form, may refer to a mixture of compounds each of which is also a polymer. The terms “a” (or “an”), “one or more” and “at least one” may be used interchangeably herein. The terms “comprising”, “including”, and “having” may be used interchangeably.

The term “about” when referring to a number means ±10%. For example, the phrase “about 35” refers to a number between and including 31.500 and 38.500. The symbol “mm” means a millimeter. The symbol “N” means newton, or kg·m·s⁻². The symbol “MPa” means megapascals or 1×10⁶ kg·m⁻¹·s⁻².

The definition of the term “device” includes a manufacture. The definition of the term “device” includes an article of manufacture. Further, the definition of the term “device” includes an article produced from raw or prepared materials by giving to these materials new forms, qualities, properties, or combinations, whether by hand labor or by machinery. The definition of the term “device” includes a machine.

The terminology related to the directions and orientation of the device of the present invention, unless indicated specifically or by usage otherwise, is consistent with the orientation as the device is designed to be worn. The term “top”, as in phrases such as “top bracket edge” or “top plate edge” refers to the portion of the device part that is closer to the head of the patient, or is in the cephalad direction. The term “bottom” as in phrases such as “bottom bracket edge” or “bottom plate edge” refers to the portion of the device part that is closer to the feet of the patient, or is in the caudal direction. The term “front”, as in phrases such as “front plate face”, refer to the portion of the device part that is further away from the body of the patient. The term “back”, as in phrases such as “back plate face”, refer to the portion of the device part that is closer to the body of the patient. The term “side”, as in phrases such as “first plate side edge” or “second plate side edge” refers to the portions of the device that is in the same transverse plane as the complementary other side, wherein the two sides are on the extreme portions of the device. The term “vertical” refers to the direction that is substantially parallel to the patient's body's longitudinal axis.

Many of the materials from which the device may be manufactured are recognized by a person skilled in the art to be polymers. The definition of the term “polymer” is broad; the definition includes a polymer, a co-polymer, plurals thereof, synthetic and well as naturally occurring polymers. For readability purposes, such polymers may be referred to by their name without the noun “polymer” appended thereto. For example, the definition of the material “ethylene vinyl acetate” includes ethylene vinyl acetate polymer, ethylene vinyl acetate polymers, ethylene vinyl acetate copolymer, and ethylene vinyl acetate copolymers.

The names of any compounds, salts thereof, compositions, or mixtures thereof refer to such compositions in their either there pure state, or as a mixture with an impurity.

The term “sharp”, as in the phrase “sharp corner”, refers to the corner where two planes or two lines intersect. Such a corner may have a radius of 0 mm. Because corners of physical objects typically have a radius greater than 0 mm, the definition of the term “sharp”, as in the phrase “sharp corner” includes a corner that has a radius of less than about 1 mm. The angle at which the two planes or two lines interest may be an acute angle, a right angle, or an obtuse angle. The term “corner” refer to where either two planes intersect or two lines intersect.

The definition of the term “ostomy” includes colostomy, ileostomy and jejunostomy. A colostomy is a surgical procedure in which an opening (stoma) is formed by drawing the healthy end of the large intestine or colon through an incision in the anterior abdominal wall and suturing it into place. The stoma, in conjunction with the attached stoma appliance, provides an alternative channel for waste to leave the body. The definition of the term colostomy includes loop colostomy, end colostomy, and double barrel colostomy.

Ileostomy is a surgical procedure which creates stoma constructed by bringing the end or loop of small intestine (the ileum) out onto the surface of the skin. Intestinal waste passes out of the stoma and is collected in an artificial external pouching system which is adhered to the skin.

Jejunostomy is a surgical procedure which creates of a fistula or a stoma through the skin at the front of the abdomen and the wall of the jejunum.

The term “ostomy pouch”, or “pouch”, refers to any type of a pouch or a bag that is worn in concert with a stoma. The ostomy pouch is used to collect waste, such as stool, urine, and mucus, from a fistula as it exits the patient's body. Examples of an ostomy pouch that may be used with the device of the present invention include a closed pouch, a drainable pouch, a two piece system, a one piece system.

The device of the present invention may be used with an ostomy pouch from most ostomy pouch manufacturers. Ostomy pouches for use with the device of the present invention may be obtained from at least some of the following ostomy product suppliers: Blanchard Ostomy Products, B. Braun Medical, CliniMed, Coloplast, ConvaTec, Cymed Ostomy Co., Dansac, Hollister, Marlen Healthcare, Marlen Manufacturing & Development Co., Nu-Hope Laboratories, Inc., Oakmed, Ostomy SuperSan, Peak Medical, Pelican Healthcare, Penlan Medican, Perfect Choice Medical Technologies, Salts Healthcare, Schena Ostomy Technologies, Inc., Securi-T USA, Trio Healthcare, and Torbot Group, Inc.

The term “ostomy catheter”, or “catheter”, refers to any type of a thin tube which allows for drainage from a stoma.

The term “patient” refers to patients who have undergone an ostomy. The term patient is an ostomate. When referring to the characteristics of the patient, such as strength, these characterizations refer to that of a typical ostomy patient, and not to the general population. The phrase “typical patient” refers to any patient within two standard deviations from the mean.

The present invention relates to a device designed to be worn by ostomy patients. This device is designed to be worn between the body of the patient and a garment of the patient. The device comprises a base plate, a bracket and a belt clip.

One of the advantages of the present invention is that it improves the patient's quality of life. The patient is able to wear his or her own clothing in a manner that is similar to those without a stoma.

Another advantage of the present invention is the ability of the wearer to insure that the stoma pouch or a catheter is not pinched during the wearing of clothing.

Still another advantage of the present invention is the light weight of the device.

Another advantage of the present invention is the ability of the wearer to pass a tubing that may be attached to the stoma pouch through the typical zippered opening located in the front of many pants, and pass liquid through the tubing while the patient stands.

The present invention relates to a device designed to be worn by an ostomy patients. The device 10 allows a passage of an ostomy pouch between a garment and a patient's body comprising (a) a base plate 20 comprising a front plate face 21, a back plate face 22, a top plate edge 23, a bottom plate edge 24, a first plate side edge 25, and a second plate side edge 26; (b) a bracket 40 comprising a linking portion 41, a first arm portion 42, and a second arm portion 43, further comprising a bracket outer surface 44, a bracket inner surface 45, a top bracket edge 46, a bottom bracket edge 47, wherein the first arm portion 42 is attached to the front plate face 21, and wherein the second arm portion 43 comprises a first snap latch portion 66 that engages a second snap latch portion 67 attached to the front plate face 21; and (c) at least one belt clip 90 attached to the top bracket edge 46 or the outer surface of the bracket 44. The device may comprise other parts in addition to ones recited in subparagraphs (a) through (c) above.

The device comprises at least the base plate, the bracket, and the belt clip. The base plate is a thin plate of material to which the bracket and the second snap latch portion are attached.

The plate comprises at least a front plate face 21, a back plate face 22, a top plate edge 23, a bottom plate edge 24, a first plate side edge 25, and a second plate side edge 26. The term “plate face” applies to either the front plate face 21, the back plate face 22, or both. The term “plate side edge” applies to the first plate side edge 25, the second plate side edge 26, or both. The term “horizontal plate edge” applies to the top plate edge 23, the bottom plate edge 24, or both.

The front plate face 21, the back plate face 22, the top plate edge 23, the bottom plate edge 24, the first plate side edge 25, and the second plate side edge 26 under one embodiment form roughly a cuboid (i.e., rectangular prism), wherein one dimension is much less than the other two dimensions.

Under one embodiment the front plate face 21 is substantially parallel to the back plate face 22. Under an alternative embodiment the thickness of the plate is varied.

Under one embodiment the plate faces are substantially rectangular, having sharp corners. Under an alternative embodiment the corners 31, 32, 33, and 34 of the plate faces are rounded. Having rounded corners mitigates the danger of the patient getting poked by the sharp corners of the plate, possibly causing unnecessary discomfort to the patients, or tearing the patient's clothing, or tearing the ostomy pouch. The rounded corners may be any size that is suitable to the device without degrading the usefulness of the device. Under an embodiment at least one of the four corners has a radius of about 1 mm to 20 mm. Under an embodiment at least one of the four corners has a radius of about 2 mm to about 10 mm. Under an embodiment at least one of the four corners has a radius of about 4 mm to about 7 mm.

Under one embodiment the base plate 20 has a plurality of holes 29 connecting the front plate surface to the back plate surface. One advantage of such holes is to provide breathability of the patient's skin through the base plate 20. Another advantage of holes 29 is the decrease of the weight of the device 10. Yet another advantage of holes 29 is the decrease of the material required and corresponding cost to manufacture the device 10.

Such holes may be any size and shape, as long as their size and shape is sufficiently consistent with the function of the base plate. A hole may be a triangular prism, a quadrilateral prism, a pentagonal prism, a hexagonal prism, an octagonal prism, and like. Alternatively, such a hole may be a circular prism (i.e., cylindrical) or an oval prism. The phrases incorporating the term “prism”, such as “hexagonal prism”, means that the hole is defined by a 2-dimensional shape (such as a hexagon) on the front plate face 21, a matching shape on the back plate face 22, and the hole wall 30 connecting these 2-dimensional shapes.

One advantage of holes being round, oval or any other shape wherein the hole wall 30 does not have any sharp concave corners, is that it is easier for the back plate 20 to be exposed to stresses without tearing the back plate.

Under one embodiment the two plate side edges when the device is in an unstressed position (i.e., when the device is not being worn) are substantially parallel to each other, and are substantially perpendicular to the horizontal plate edges. Under another embodiment the first plate side edge 25 leans in, so that the top plate edge 23 is shorter than the bottom plate edge 24. Under another embodiment the first plate side edge 25 leans out, so that the top plate edge 23 is longer than the bottom plate edge 24.

Under one embodiment the second plate side edge 26 leans in, so that the top plate edge 23 is shorter than the bottom plate edge 24. Under another embodiment the second plate side edge 26 leans out, so that the top plate edge 23 is longer than the bottom plate edge 24.

Under one embodiment the top plate edge 23 is straight. Under an alternative embodiment the top plate edge 23 comprises a concavely curved portion 27. The phrase “concavely curved portion” means that instead of the edge being straight, the edge runs in a portion of the top plate edge closer towards the geometric center of the base plate 20. The concavely curved portion can be of any deviation from a straight line, provided that its lowest point does not cross the imaginary straight line 28 connecting the uppermost point of the base of the first arm portion 54 with the uppermost point of the base of the second snap latch portion 55. The concavely curved portion is between about 50% to about 95% of the plate edge 23.

The advantage of having the concavely curved portion 27 is that it provides a space for the ostomy pouch attachment to the patient's body. The concavely curved portion 27 may also more comfortable to wear than for a comparable device wherein the top plate edge 23 is straight.

The size of the base plate is selected so that it matches the size of the bracket, which in turn is selected to match the size of the ostomy pouch. For use with a typical ostomy pouch, the length of the base plate (i.e., the length as measured along the horizontal edge), is between about 100 mm and about 200 mm. Under some embodiments the length of the base plate is between about 130 mm and 180 mm.

For use with a typical ostomy pouch, the height of the base plate (i.e., the length as measured along the vertical edge), is between about 60 mm and about 130 mm. Under some embodiments the length of the base plate is between about 80 mm and 100 mm.

Depending on the material, according to some embodiments the base plate is flexible. When the bracket 40 is unlatched from the base plate 20 the base plate, the base plate 20 folds away from the bracket.

The device of the present invention comprises at least the base plate, the bracket, and the belt clip. The bracket is a C-shaped piece that is permanently attached to the base plate 20 on one side, and attaches on the other side by a latch. The latch may be opened and closed.

The bracket 40 comprises a linking portion 41, a first arm portion 42, and a second arm portion 43. The linking portion 41 links the first arm portion 42 to the second arm portion. The term “portion” in the phrases “linking portion” and “arm portion” refers to the portion of the bracket 20.

The bracket 40 also comprises a bracket outer surface 44, and an bracket inner surface 45, a bracket top edge 46, and a bracket bottom edge 47. The bracket top edge 46 spans all portions of the bracket, including the linking portion 41, the first arm portion 42, and the second arm portion 43. The bracket bottom edge 47 spans all portions of the bracket, including the linking portion 41, the first arm portion 42, and the second arm portion 43.

The bracket outer surface 44 spans all portions of the bracket, including the linking portion 41, the first arm portion 42, and the second arm portion 43. The bracket inner surface 45 spans all portions of the bracket, including the linking portion 41, the first arm portion 42, and the second arm portion 43.

The linking portion outer surface 48 is the part of the bracket outer surface 44 that spans the linking portion 41. The first arm portion outer surface 49 is the part of the bracket outer surface 44 that spans the first arm portion 42. The second arm portion outer surface 50 is the part of the bracket outer surface 44 that spans the second arm portion 43. The linking portion inner surface 51 is the part of the bracket inner surface 45 that spans the linking portion 41. The first arm portion inner surface 52 is the part of the bracket inner surface 45 that spans the first arm portion 42. The second arm portion inner surface 53 is the part of the bracket inner surface 45 that spans the second arm portion 43.

Under one embodiment the linking portion 41 and the first arm portion 42 meet at a sharp corner, and form an angle that is substantially a right angle. The bracket outer surface 44 may have a sharp break, so that the linking portion outer surface 48 comes to a sharp convex corner with the first arm portion outer surface 49.

Under an alternative embodiment, the transition of the bracket outer surface from the linking portion 41 to the first arm portion 42 is gradual. The intersection of the linking portion outer surface 48 and the first arm portion outer surface 49 form a round 57.

Under one embodiment the bracket inner surface 45 has a sharp break, so that the linking portion inner surface 51 comes to a sharp concave corner with the first arm portion inner surface 52.

Under an alternative embodiment, the transition of the bracket inner surface 45 from the linking portion 41 to the first arm portion 42 is gradual. The intersection of the linking portion inner surface 51 and the first arm portion inner surface 52 forms a fillet 56.

Under one embodiment the linking portion 41 and the second arm portion 43 meet at a sharp corner edge, and form an angle that is substantially a right angle. The bracket outer surface 44 may have a sharp break, so that the linking portion outer surface 48 comes to a sharp convex corner with the second arm portion outer surface 50.

Under an alternative embodiment, the transition of the bracket outer surface 44 from the linking portion 41 to the second arm portion 43 is gradual. The intersection of the linking portion outer surface 48 and the second arm portion outer surface 50 form a round 59.

Under one embodiment the bracket inner surface 45 has a sharp break, so that the linking portion inner surface 51 comes to a sharp concave corner with the second arm inner portion surface 53.

Under an alternative embodiment, the transition of the bracket inner surface 45 from the linking portion 41 to the second arm portion 43 is gradual. The intersection of the linking portion inner surface 51 and the second arm portion inner surface 53 form a fillet 58.

Under one embodiment, the first arm portion 42 and base plate 20 meet at a sharp corner, and form an angle that is substantially a right angle. The first arm portion outer surface 49 has a sharp break where it meets the front plate face 21. Under an alternative embodiment, the first arm portion outer surface 49 forms a fillet 60 where it meets the front plate face 21.

Under one embodiment the first arm portion inner surface 52 has a sharp break where it meets the front plate face 21. Under an alternative embodiment, the first arm portion inner surface 52 forms a fillet 61 where it meets the front plate face 21.

The second snap latch portion 67 is attached to the front plate face 21. It is positioned so that it can engage with the first snap latch portion 66. When the first snap latch portion and the second snap latch portion are snapped together, the flexible base plate is either flat, or exhibits a slight curvature 35. Such a curvature is present so that the device as worn by the patient is able to contour itself along the body of the patient. The second snap latch portion 67 comprises a second snap latch portion outer surface 73, and a second snap latch portion inner surface 74.

Under one embodiment the second snap latch portion outer surface 73 and front plate face 21 meet at a sharp corner, and form an angle that is substantially a right angle. The second snap latch portion outer surface 73 has a sharp break where it meets the front plate face 21. Under an alternative embodiment, the second snap latch portion outer surface 73 forms a fillet 63 where it meets the front plate face 21.

Under one embodiment the second snap latch portion inner surface 74 has a sharp break where it meets the front plate face 21. Under an alternative embodiment, the second snap latch portion inner surface 74 forms a fillet 62 where it meets the front plate face 21.

The device 10 of the present invention may have any combinations of the sharp corners, rounded outer corners and filleted inner corners.

It is generally advantageous for the outer corners to be rounded and the inner corners to be filleted. One of the possible advantages is that the device or the bracket is able to distribute the stresses along gradually transitioning portions more easily then if the inner corners would be sharp. Another possible advantage is that the device with filleted corner do not tear as easily as comparable device with sharp corners. Still another possible advantage is that the rounded outer corners do not poke garments or patients as sharp convex corners would. Yet another possible advantage of that the fillet inner corners do not restrict the movement of ostomy pouch or pinch the ostomy pouch as a sharp inner corner may.

The second arm portion 43 comprises a first snap latch portion 66. The first snap latch portion 66 is designed to mate with the second snap latch portion 67. The first snap latch portion 66 and the second snap latch portion 67 together form a snap latch.

The snap latch is comprised of two snap latch portions which connect to create a temporary bond between the two snap latch portions. Under one embodiment, one snap latch portion is a male snap latch portion, and the other snap latch portion is a female snap latch portion.

FIG. 6a shows a detail of one example of a latch when viewed from the top (or alternatively, from the bottom), wherein the male snap portion 70 is attached to the plate 20, and the second arm portion 43 comprises the female snap latch portion 80.

FIG. 6b shows a detail of another example of the latch when viewed from the top (or alternatively, from the bottom), wherein the female snap portion 80 is attached to the plate 20, and the second arm portion 43 comprises the male snap portion 70.

The male snap latch portion 70 comprises a ridge, which viewed from the top comprises a neck portion 71 and a head portion 72. The head portion 72 is generally bulbous and is thicker than the neck portion 71. The female snap latch portion 80 comprises an inner ridge 81 and an outer ridge 82 and a valley 83 between the inner ridge 81 and the outer ridge 82.

To close the snap latch, the male snap latch portion 70 is thrust into the valley 83 until the head 72 snaps into place.

The force needed to snap the male snap latch portion 70 into the female snap latch portion 80 should be no greater than the patient can comfortably exert. The snap generally occurs when the device is placed in the wearing position, and the patient then snaps the latch in place.

Under one embodiment the force needed to snap the snap latch shut is no more than the palmar pinch of the typical patient. Under another embodiment the force needed to snap the snap latch shut is no more than the key pinch of the typical patient. Under yet another embodiment the force needed to snap the snap latch shut is no more than the tip pinch of the typical patient. The force need to close the snap latch shut is less than about 40 N. Under an alternative embodiment the force need to close the snap latch shut is less than about 20 N.

The force needed to open the latch is not more the force that the patient can comfortably exert to open the latch. Under one embodiment the force needed to open the latch is less than about 50 N. Under another embodiment the force needed to open the latch is less than about 25 N.

The force needed to open the latch is not less than the force that an active patient will experience during the patient's routine activities. Under one embodiment the force needed to open the latch is more than about 2 N. Under another embodiment the force needed to open the latch is more than about 5 N.

The two snap latch portions may be manufactured from the same material or from a different material, as long as at least one of them is flexible enough so that the male snap latch portion 70 snaps reversibly into the female snap latch portion 80.

The device may have the latch on either side of the base plate. The device may be a left swinging device or a right swinging device.

In FIG. 1 through FIG. 5 the first arm portion 42 is attached close the right side of the base plate 20, and the second snap latch portion 67 is attached close to the left side of the base plate 20. Under an alternative embodiment, the first arm portion 42 is attached close the left side of the base plate 20, and the second snap latch portion 67 is attached close to the right side of the base plate 20.

Under one embodiment the ridge of the male snap latch portion along the vertical direction is uniform. Under another embodiment the head diameter varies along the vertical direction. Under one embodiment the size of the head portion 72 is larger in the top and bottom portions of the ridge, and smaller in the central part of the ridge. Under an alternative embodiment the size of the head portion 72 is smaller in the top and bottom portions of the ridge, and larger in the central part of the ridge.

Under one embodiment the male snap latch portion 70 comprises a fin 75. The fin 75 is a projection from the male snap latch portion 70 that when the snap latch is closed, the fin 70 fits into slot 84 in the female snap latch portion 80.

Under one embodiment, the slot 84 is located on the outer ridge 82 and the fin is located on the male snap latch outer surface 77. Under another embodiment the slot 84 is located on the inner ridge 81 and the fin is located on the male snap latch inner surface 76.

There may be a plurality of fins on the ridge of the male snap latch portion 70 and a complementary number of slots in the female snap latch portion 80.

The presence of the fin or fins helps to stabilize the closure of the snap latch. Further, the fin or fins prevents the vertical movement of the male snap latch portion 70 with respect to the female snap latch portion 80.

The device of the present invention comprises at least the base plate, the bracket, and at least one belt clip. The belt clip 90 is attached to the bracket on either the bracket top edge 46, to the bracket outer surface 44.

Under one embodiment the device comprises one belt clip. Under another embodiment the device comprises two belt clips. Under yet another embodiment the device comprises three or more belt clips.

The belt clip comprises a belt clip outer surface 91, a belt clip inner surface 92, a connecting portion 93, a first belt clip side edge 94, a second belt clip side edge 95, and a bottom belt clip edge 96.

Under one embodiment the two belt clip side edges are substantially parallel to each other, and are substantially perpendicular to the connecting portion 93. Under another embodiment the first belt clip side edge 94 leans in, so that the connecting portion 93 is shorter than the belt clip bottom edge 96. Under another embodiment the first plate side edge 94 leans out, so that the connecting portion 93 is longer than the belt clip bottom edge 96.

Under another embodiment the second belt clip side edge 95 leans in, so that the connecting portion 93 is shorter than the belt clip bottom edge 96. Under another embodiment the second plate side edge 95 leans out, so that the connecting portion 93 is longer than the belt clip bottom edge 96.

The belt clip bottom edge 96 may be straight or it may be curved.

Under one embodiment the corners 97 and 98 formed by the intersection of the belt clip bottom edge 96 with the first belt clip edge 94 and the second first belt clip edge 96, respectively, are sharp corners. Under an alternative embodiment the corners 97 and 98 are rounded. Having rounded corners mitigates the danger of the patient getting poked by the sharp corners of the belt clip, possibly causing unnecessary discomfort to the patients, tearing the patient's clothing, or tearing the ostomy pouch. The rounded corners may be any size that is suitable to the device without degrading the usefulness of the device. Under an embodiment at least one of the two corners has a radius of about 1 mm to 20 mm. Under an embodiment at least one of the two corners has a radius of about 2 mm to about 10 mm. Under an embodiment at least one of the two corners has a radius of about 4 mm to about 7 mm.

The structure of the device 10 may be modular or unibody. For a modular construction, the bracket 40 may be formed from plastic, metal, wood, or other rigid material or semi-rigid material. The base plate 20 may be formed from a flexible material such as rubber, leather, or plastic; and the belt clip may be formed from plastic, metal, wood, or other rigid material. Under one embodiment the plastic, metal, wood, or other rigid material that the bracket is formed from is the same as the plastic, metal, wood, or other rigid material that forms the belt clip. Under another embodiment the plastic, metal, wood, or other rigid material that the bracket is formed from is different from the plastic, metal, wood, or other rigid material that forms the belt clip. The back plate, the bracket, and the belt clip may be joined by any suitable means, including fasteners such as screws, nuts and bolts, rivets, and nails; glue, welding, or any other commercially available unitizing method.

Under one embodiment the device has a unibody structure. The term “unibody” means that the entire device, or essentially the entire device, or the majority of the entire device is made from the same material. Alternatively, the term “unibody” means that the base plate, the bracket and the belt clip are made from the same material. A unibody device to which other parts or additives (such as a coating) are added is a unibody device.

The unibody device is composed of a single material which has uniform chemical and physical properties. The material used is rigid enough that the device holds its shape during patients everyday activities to allow for the ostomy pouch to be passed therethrough. However, the material used is also flexible enough that the latch can be opened and closed. Further, the material is flexible enough that the bracket and the base plate can be separated to a sufficient degree so that the ostomy pouch can be placed therein or removed therefrom.

The flexibility and rigidity may be quantified by the elastic modulus of the material. The elastic modulus may be evaluated by ISO 527 or a similar standard method. Under one embodiment the phrase “ISO 527” refers to the latest revision of the standard. Under another embodiment the phrase “ISO 527” refers to any of the standards carrying the ISO 527 designation that is applicable to determine the elastic modulus of the material to form the device of the present invention. Such standards may include ISO 527-1, ISO 527-2, such as ISO 527-1:1993, ISO 527-1:1993/Amd 1:2005, ISO 527-1:1993/Cor 1:1994, ISO 527-1:2012, ISO 527-2:1993, ISO 527-2:1993/Cor 1:1994, ISO 527-2:1996 E, and like.

Under one embodiment the elastic modulus of the material from which the device of the present invention is formed is between about 10 megapascals (MPa) and about 400 MPa as measured by ISO 527-1, -2. Under another embodiment, the elastic modulus of the material from which the device of the present invention is formed is between about 20 MPa and about 200 MPa as measured by ISO 527-1,-2. Under yet another embodiment, the apparent bending modulus of the material from which the device of the present invention is formed is between about 50 MPa and about 100 MPa as measured by ISO 527-1,-2.

The surface properties of the material from which the device is formed may also be important. Such properties include hardness. Hardness of the material may be evaluated by ISO 868 or a similar standard method. Under one embodiment the phrase “ISO 868” refers to the latest revision of the standard. Under another embodiment the phrase “ISO 868” refers to any of the standards carrying the ISO 868 designation, including ISO 868:2003, ISO 868:1985, and like.

Under one embodiment the hardness of the material from which the device is formed is between about 30 units and about 90 units as measured by ISO 868 under Shore A scale. Under another embodiment the hardness of the material from which the device is formed is between about 40 units and about 80 units as measured by ISO 868 under Shore A scale. Under still another embodiment the hardness of the material from which the device is formed is between about 50 units and about 60 units as measured by ISO 868 under Shore A scale.

Additional qualities which the device of the present invention also exhibits include low friability, static discharge mitigation, and good antimicrobial properties.

Under one embodiment the material from which the unibody device of the present invention is formed and that exhibits such properties is a polymer. The device may be formed by injection molding, or by other means.

Suitable polymers include ethylene vinyl acetate, ethylene-vinyl rubber, polyethylene, polyvinyl chloride, polyurethane, rubber, polypropylene, polyethylene terephthalate, polyethylene-octene, polyethylene-butene, high density polyethylene, low density polyethylene, polyethylene acrylonitrile butadiene styrene, high impact polystyrene, polypropylene, polycarbonate, acrylonitrile butadiene styrene, polyamide, polyethersulfone, a co-polymer of any one or more of the preceding, and a mixture thereof.

Suitable polymers also include ethylene vinyl acetate, polyethylene-octene and polyethylene-butene.

Under one embodiment the polymer, co-polymer, or mixtures thereof further comprises other ingredients that improve the physical properties of the material, chemical properties of the material, and/or aesthetic properties of the material. Possible ingredients include a pigment, an antimicrobial ingredient, a lubricating agent, a nucleating agent, an antioxidant, an antistatic agent, and any combinations thereof.

At least some of these ingredients need to be thermally stable up to the processing temperatures of the material. Such ingredients should be safe for the patient to use, comply with applicable health and safety regulations.

Under one embodiment such ingredients should be prevented from migrating onto the skin or clothing of the patient. Under an alternative embodiment some ingredients (such as antimicrobial agents) migrate to the surface of the device.

The pigment used as an ingredient may be used to provide a color to the formed device. Such colors may have an esthetic purpose. For example, the color of the device may be black or brown to match a belt on the patient's pants, blue to match the denim, or any other color to visually mask the device vis-à-vis the color of the clothing. Alternatively, the color may have a functional purpose, such color-coding, for example, different colors may be indicative of the device's rigidity, elastic modulus, hardness, or size.

Examples of suitable pigments which can be used in accordance with this invention include finely divided rutile titanium dioxides, lithopone, antimony oxide, barytes, diatomaceous silica, clay, mica, iron oxides, cadmium red, chromium oxide clay, mica, iron oxides, cadmium red, chromium oxide, green, lampblack, carbon black, mineral black and luminescent pigments.

Further examples of pigments include Yellow H9G; Yellow H4G, Yellow H2G, Yellow HG 01, Yellow HG, Yellow H2GR, Yellow HGR, Yellow HR 02, Yellow HR, Yellow H3R, Orange H4G1 01, Orange H2GL, Orange GRL, Orange 6RL, Scarlet 4RF, Red HGR, Red B, Red D3G, Red HB, Red 3B, Red BNP, Red HF4B, Red E3B, Red E5B, Yellow Hl0G, Pink E, Yellow GG, Pink E 01, Yellow 3GP, Violet ER, Yellow GR, Violet BLP, Yellow H2R, Violet RL, Orange GPS, Blue A4R, Orange RL, Blue A2R, Red HFG, Blue BG, Red LG, Green GNX, Red LC, Brown HFR, Red BB, Brown RL, Fire Red 3RLP, Red E4G, Red HF2B, Red F5RK, Red 2BN, Red P2B, Carmine HF4C, Carmine HF3C, Rubine L4B, Bordeaux HF3A, Blue AN, (all available from Clariant International Ltd, Rothausstrasse 61, 4132 Muttenz, Switzerland), their chemical equivalents, and any mixture comprising such chemical equivalents, and any mixture of any two or more of the preceding.

Controlling the hygiene and growth of microorganisms in areas of body that are in the proximity to being exposed to bodily fluids is essential. The device of the present invention is constructed to handle such a demanding environment. Examples of such construction are disclosed below in various embodiments, some of which may be combined with each other.

Under one embodiment the device of the present invention is constructed from a solid material, the surface of which can be easily be disinfected by disinfectants, such as household bleach, to control the growth of microorganisms.

Under another embodiment the device of the present invention is constructed from a closed cell foam material, the surface of which can be easily be disinfected by disinfectants, such as household bleach, to control the growth of microorganisms.

Under yet another embodiment the device of the present invention is constructed from a skinned foam material, the surface of which can be easily be disinfected by disinfectants, such as household bleach, to control the growth of microorganisms.

In some embodiments anti-microbial agents may be added to the material during the manufacture process. The antimicrobial agent that is used as an ingredient in the material functions to kill, mitigate or otherwise control the growth of microorganisms. More specifically, the addition of anti-microbial agents may be useful in decreasing the amount of microbes present on the device, decreasing the probability that microbes reside on the device, and/or decreasing the probability that microbes form a biofilm on the device.

Antimicrobial agents include disinfectants, biostatics, antifungals, antibacterials, bactericidal agents, bacteriostatic agents. Under one embodiment the microbiological agents are polymeric antimicrobial agents. Any antimicrobial agent that is usable on plastic materials may be used as the antimicrobial agent in the present invention, as long as the activity of the antimicrobial agent is not significantly decreased during the formation of the material.

In some embodiments, the anti-microbial agent or substance can be an anti-microbial metal. Non-limiting examples of metals with anti-microbial properties are silver, gold, platinum, palladium, copper, iridium, tin, antimony, bismuth, zinc cadmium, chromium, and thallium.

Further examples of anti-microbial agents include, inorganic particles such as barium sulfate, calcium sulfate, strontium sulfate, titanium oxide, aluminum oxide, silicon oxide, zeolites, mica, talcum, and kaolin.

Still further examples of antimicrobial agents include 4-aminobenzoic acid, salicylic acid, hydroxybenzoic acid, triclosan, and chlorhexidine hexametaphosphate.

Examples of lubricating agents or sliding agent include the compounds of starches, stearates, glycerols such as glycerol monostearate, cis-13-docosenoamide, 13-docosenamide, and mixtures of these compounds.

The nucleation agent can be selected from the compounds of sodium benzoate, 2,2-methylene-bis-4,6-di-tert-butylphenyl lithiophosphate, 1,3:2,4-bis (3,4-dimethylbenzylidene)sorbitol, magnesium silicate talc, a salt of 6-oxide-2,4,8,10-tetrakis (1,1-dimethylethyl)-6-hydroxy-12H-dibenzo[d,g][1,3,2]dioxaphosphocin, MILLAD 3988i, HPN-20E, salt of cyclohexanedicarboxylic acid, zinc stearate, HPN-600EI, and mixtures thereof.

The antioxidant agent and/or an antistatic agent can be selected from the phenol antioxidant compounds such as tetrakis[methylene-3(3′,5′-di-tert-butyl-4′-hydroxyphenyl) propionate]methane, tris(2,4-di-tert-butylphenyl) phosphite, 1,3,5-trimethyl-2,4,6-tris(3,5-di-tert-buty-4-hydroxybenzyl)benzene, pentaerythritol tetrakis[3-(3,5-di-tert-butyl-4-hydroxyphenyl) propionate], and mixtures thereof.

The antistatic agent can be selected from the mono- and/or diglyceride compounds with a C₁₄ to C₁₈ carbon atom chain, and mixtures of these compounds.

Under one embodiment at least a part of the surface of the device of the present invention is coated. The device may comprise several coats or coatings of portion of the device.

Such a coating may be used to protect the device from oxidation, from UV radiation, from the everyday wear and tear, or any other events that may degrade the material.

Such a coating may be used to increase or decrease the slipperiness of the surface of the device. Under an embodiment at least a portion of the outer surface of the belt clip is coated with a slippery material to decrease the friction of the outside surface with a garment worn by the patient. Under an embodiment at least a portion of the inner bracket surface is coated with a slippery material to decrease the friction of the inner bracket surface with the ostomy pouch. Under an embodiment at least a portion of the front plate face is coated with a slippery material to decrease the friction of the front plate face with the ostomy pouch.

Under an embodiment at least a portion of the outer bracket surface is coated with a slip-resistant material to increase the friction of the outside surface of the bracket with the garment onto which the device is clipped. Under an embodiment at least a portion of the inside belt clip surface is coated with a slip-resistant material to increase the friction of the outside surface of the belt clip with the garment onto which the device is clipped.

The coating may also be used for functional or aesthetic purposes. The coating may disguise the device as a part of an article of apparel. The coating may disguise the device so that it appears as an holder for an entertainment electronic device.

Under one embodiment the coating comprises a microbial agent. Examples of antimicrobial agents include those listed above.

The device of the present invention may further comprise a label, such as an adhesive label. The device may have more than one label. A label may be affixed permanently or temporarily. A label may be standardized, or it may be personalized.

Such a label may be used to communicate important information to the patient such as instructions on wearing the device, reminders to the patient, or warnings related to the use of the device.

A label my list information trademark information, a name of the device, a name and place of business of the manufacturer, packer, or distributor, date of manufacturer. A label may carry information as required by the U.S. Food and Drug Administration, the U.S. Consumer Protection Agency, the U.S. Patent Office, the U.S. Customs and Border Protection Agency, or any other U.S. governmental agency, or any corresponding non-U.S. sovereign national, regional, provincial, territorial, or state, agency. The label may list patent numbers, trademarks, website information, the name of the patent attorney, and other relevant intellectual property information.

A label may be used to mitigate any manufacturing defects or attributes, such a witness marks or parting lines.

A label may be individualized. An individualized label may be used to communicate vital information to emergency personnel about the patient's health condition.

A label may be partially blank or wholly blank so that it can be used by the patient to take notes, such as the time of the last stoma pouch change, and like.

The device of the present invention has been specifically designed to be worn by an ostomate patient 110 between the patient's skin and clothing to allow for a free passage of the pouch.

Because a stoma 111 may be situated anywhere on the abdomen, ostomate patients have traditionally had to wear pants, skirts and other lower body garments in a way that would not pinch the ostomy pouch 112. Patients wore their pants 130, skirts and other lower body garments high, so that the top of the pants 131, skirts and other garments were around the waist transverse plane 120, above the upper hips, or above the belly button 115.

With the device of the present invention, the patient 110 is able to wear the patient's pants 130, skirt or other lower body garment at the customary height. This customary height of the top of the pants, skirt or other lower body garment, approximated by the imaginary transverse plane 121, is between the upper hip (i.e., a convex feature telegraphed by the anterior superior ilac spine) 113, and the lower hip (i.e., the convex feature telegraphed by the greater trochanter) 114.

The device is designed to be attached to a portion of the garment by several means, such as by a belt clip 90. The device may be attached by the belt clip 90 to an edge the garment or a fold in the garment, such as the top of the pants 131.

Under one embodiment the device attaches to the top of pants 131, skirt or another article of clothing that the patient wears on a lower portion of the patient's body. The device 10 may be worn with the base plate 20 and the bracket on inside of the clothing, with the belt clip 90 on the outside of the clothing. The edge of the clothing is slipped between the bracket and the belt clip.

The edge of the clothing to which the belt clip attaches may be reinforced with a belt, such as a clothing belt. Such a belt may a typical belt that is used to support the wearer's pants.

Under another embodiment the device is worn with a belt without any clothing edge, such as a garter belt that is worn around waist.

The device of the present invention is designed so that the ostomy pouch when worn is not pinched or restricted by the clothing. When the pouch and the device are worn, the pouch is located between the bracket and the base plate. The pouch is worn loosely, so that the pouch moves freely along the longitudinal direction, as the patient moves.

The device of the present invention is also designed to work with a catheter. Many patients in addition to, or instead of, using pouches that are attached to the stoma to collect waste, use a catheter that connects the stoma with a larger bag. The larger bag may be carried strapped to the patient's leg. Under this embodiment, the patient wears the device attached to the top of the patient's pants, skirt or another article of clothing, in a similar fashion as that described above, wherein the ostomy catheter connecting the stoma with a leg pouch, is passed between the front face of the base plate and the bracket.

One of the problems of wearing pants high above the stoma is that it is difficult to pass a waste tube attached to the bottom of the pouch without taking off the pants. The device of the present invention is also designed to aid in emptying the stoma pouch by a waste tube without taking the patient's pants. The waste tube from the bottom of the stoma pouch may be passed discretely through the typical zippered opening located in the front of many pants, and pass urine or other liquid waste through the waste tube while the patient remains in the standing position.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. A device (10) for allowing a passage of an ostomy pouch between a garment and a patient's body comprising
(a) a base plate (20) comprising a front plate face (21), a back plate face (22), a top plate edge (23), a bottom plate edge (24), a first plate side edge (25), and a second plate side edge (26);
(b) a bracket (40) comprising a linking portion (41), a first arm portion (42), and a second arm portion (43), further comprising an outer bracket surface (44), an inner bracket surface (45), a top bracket edge (46), a bottom bracket edge (47), wherein the first arm portion (42) is attached to the front plate face (21), and wherein the second arm portion (43) comprises a first snap latch portion (66) that engages a second snap latch portion (67) attached to the front plate face (21); and
(c) at least one belt clip (90) attached to the top bracket edge (46) or the outer surface of the bracket (44).

2. The device of claim 1, wherein the first snap latch portion (66) is a female snap latch portion (80), and the second snap latch portion (67) is a male snap latch portion (70).

3. The device of claim 1, wherein the first snap latch portion (66) is a male snap latch portion (70), and the second snap latch portion (67) is a female snap latch portion (80).

4. The device of any of claims 1 to 3, wherein the top plate edge (32) comprises a concavely curved portion (27).

5. The device of any of claims 1 to 4, further comprising linking portion outer surface (48), a first arm portion outer surface (49), a second arm portion outer surface (50), a linking portion inner surface (51), a first arm portion inner surface (52), and a second arm portion inner surface (53); further comprising:
(d)(i) a fillet (56) formed by the linking portion inner surface (51) and the first arm portion inner surface (52),
(ii) a round (57) formed by the linking portion outer surface (48) and the first arm portion outer surface (49),
(iii) a fillet (58) formed by the linking portion inner surface (51) and the second arm portion inner surface (53),
(iv) a round (59) formed by the linking portion outer surface (48) and the second arm portion outer surface (50),
(v) a fillet (60) formed by the front plate face (21) and the first arm portion outer surface (49),
(vi) a fillet (61) formed by the front plate face (21) and the first arm portion inner surface (52),
(vii) a fillet (62) formed by the front plate face (21) and the second snap latch portion inner surface (74),
(viii) a fillet (63) formed by the front plate face (21) and the second snap latch portion outer surface (73), or
(ix) any combination thereof

6. The device of any of claims 1 to 5, wherein the base plate (20) comprises a plurality of holes (29).

7. The device of any of claims 2 to 6, the male snap latch portion (70) comprises a fin (75) and the female snap latch (80) comprises a slot (84) that engages the fin.

8. The device of any of claims 1 to 7, wherein at least a portion of a surface of the device is covered by a coating.

9. The device of any of claims 1 to 8, wherein the device further comprises a label.

10. The device of any of claims 1 to 9, wherein the device has a unibody structure.

11. The device of claim 10, wherein the device is formed by injection molding.

12. The device of claim 10 wherein the device is comprised of material selected from the group consisting of ethylene vinyl acetate, ethylene-vinyl rubber, polyethylene, polyvinyl chloride, polyurethane, rubber, polypropylene, polyethylene terephthalate, high density polyethylene, low density polyethylene, polyethylene acrylonitrile butadiene styrene, high impact polystyrene, polypropylene, polycarbonate, acrylonitrile butadiene styrene, polyamide, polyethersulfone, a co-polymer of any one or more of the preceding, and a mixture thereof.

13. The device of claim 12, wherein the material further comprises an ingredient selected from the group consisting of a pigment, an antimicrobial agent, a lubricating agent, a nucleating agent, an antioxidant, an antistatic agent, and any mixture thereof

14. The device of claim 10, wherein the hardness of the material from which the device is formed is between about 30 units and about 90 units as measured by ISO 868 under Shore A scale.

15. The device of claim 10, wherein the elastic modulus of the material from which the device is formed is between about 20 MPa and about 200 MPa as measured by ISO 527.
